(19) United States
(12) Patent Application Publication
(10) Pub. No.: US 2017/0065805 A1
(43) Pub. Date: Mar. 9, 2017

(54) THERAPEUTIC TROCAR/INSERTER AND METHOD OF USE

(71) Applicant: THE SOTOPELLE GROUP, LLC, Scottsdale, AZ (US)

(72) Inventor: Gino TUTERA, Paradise Valley, AZ (US)

(21) Appl. No.: 15/257,576

(22) Filed: Sep. 6, 2016

Related U.S. Application Data
(60) Provisional application No. 62/214,672, filed on Sep. 4, 2015.

(51) Int. Cl. A61M 37/00; A61B 17/34

(52) U.S. Cl. CPC A61M 37/0069; A61B 17/3417; A61B 2017/3454; A61B 2560/0425

(57) ABSTRACT

Systems and methods for a trocar system including a trocar with a body portion defining a loading chamber, an ergonomically shaped handle portion, and an incising portion that defines a beveled tip. A lumen is formed through the body portion, the handle portion, and the incising portion. An obturator includes a tip, a shaft, and an obturator handle. The shaft is sized to be received within the lumen.

### CROSS-REFERENCE TO RELATED APPLICATIONS

This Application claims benefit of U.S. Provisional Application 62/214,672 filed on Sep. 4, 2015, and incorporated herein by reference.

### BACKGROUND

Field

Embodiments of the present invention generally relate to a system for subcutaneous delivery of hormone pellet therapy.

Description of the Related Art

Hormone pellet therapy may be used to deliver hormones into a patient's body. Hormone replacement therapy can include estradiol and testosterone for females and testosterone for males. Typical trocars used for the delivery of implants or pellets require pressure from the users forearm to advance the trocar into subcutaneous tissue linearly with no rotation. The resultant dissection is a sharp cut and occasional bruising. If an obstruction is encountered, the user applies additional force with the forearm to drive the trocar through the obstruction causing additional tissue damage.

Systems and methods for providing a blunt dissection with less tissue trauma and more dexterity are desirable.

### SUMMARY

Trocar systems and methods are provided and include a trocar including a body portion defining a loading chamber, a ergonomically shaped handle portion, and an incising portion that defines a beveled tip. A lumen is formed through the body portion, the handle portion, and the incising portion. An obturator includes a tip, a shaft, and an obturator handle. The shaft is sized to be received within the lumen.

A method is provided for a user introducing a hormone pellet into a patient at an implant location using a trocar system that includes a trocar including a body portion defining a loading chamber, a ergonomically shaped handle portion, and an incising portion that defines a beveled tip. A lumen is formed through the body portion, the handle portion, and the incising portion. An obturator includes a tip, a shaft, and an obturator handle. The shaft is sized to be received within the lumen. The method includes anesthetizing the implant location, incising the implant location with a wound, inserting the trocar into the wound using only force from the user's forefinger and thumb and rotating motion, loading a pellet into the loading chamber, loading the obturator into the lumen behind the pellet, and dispensing the pellet into the wound using the obturator.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a pictorial view of a trocar system.

FIG. 2 is an exploded pictorial view of the trocar system of FIG. 1.

FIG. 3 is a section view of the trocar system of FIG. 1 taken along line 3-3 of FIG. 1.

FIG. 4 is a section view of the trocar system of FIG. 1 taken along line 4-4 of FIG. 1.

FIG. 5 is a side elevation view of an alternative obturator.

FIG. 6 is a side elevation view of another obturator.

FIG. 7 is a pictorial view of an alternative trocar.

FIG. 8 is a side elevation view of the trocar of FIG. 7.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present invention is described with reference to the attached figures. The figures are not drawn to scale and they are provided merely to illustrate embodiments of the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods or features. In other instances, well-known structures or operation are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

The word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is if, X employs A; X employs B; or X employs both A and B, then “X employs A or B” is satisfied under any of the foregoing instances.

The present invention concerns implementing systems and methods for inserting implants of hormone pellets into the subcutaneous tissue of a patient. While the specific implementations described herein are directed to a trocar and obturator for dissection and delivery of hormone pellets, it is envisioned that the systems and methods described herein can be used with minor modification for insertion of other implants, without limitation.

SOTTOPELLE (™) hormone replacement therapy (“HRT”) using Hormone Pellet Therapy is a highly effective form of HRT. This treatment method is based on prescribing the right kind of hormone (e.g., biologically identical), in the correct amounts (e.g., determined through testing & proper analysis of the results), using the right delivery system (e.g., pellets).

The present disclosure provides a system and method for delivering a predetermined amount of the right kind of hormone with a trocar system.

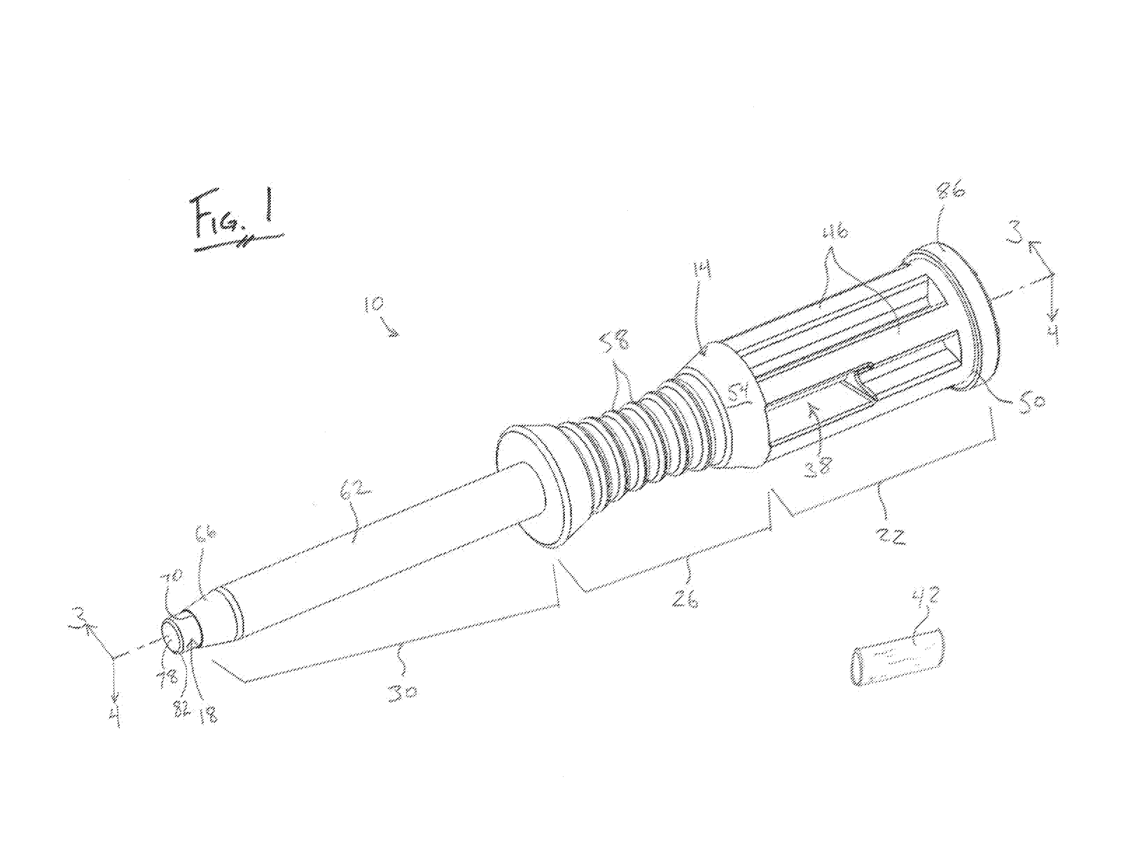

FIG. 1 shows an exemplary trocar system 10 that includes a trocar 14 and an obturator 18. The trocar 14 includes a body portion 22, a handle portion 26, and an incising portion 30. A lumen 34 (see FIG. 3) is defined through the trocar 14 and sized to receive the obturator 18. The body portion 22 includes an aperture in the form of a loading chamber 38, which is sized to receive a pellet 42, structural ribs 46, and an obturator flange 50. The loading chamber 38 provides communication to the lumen 34, such that pellets 42 may be introduced into the lumen 34 as will be discussed in greater detail below.

The handle portion 26 includes a gripping surface 54 with texture in the form of ribs 58 and defines an ergonomic shape in the form of a hyperbolic profile that allows a user to grip the handle portion 26 during use with less muscle fatigue.

With reference to FIG. 3, the incising portion 30 (FIG. 1) includes a cannula 62 and a beveled tip 66 that defines a circular opening 70 with a penetrating surface defined in a plane A perpendicular to an axis B of the lumen 34. The beveled tip 66 defines a penetrating angle C of about ten degrees (10°). In other embodiments, the penetrating angle C may be between about five degrees (5°) and about 20 degrees (20°).

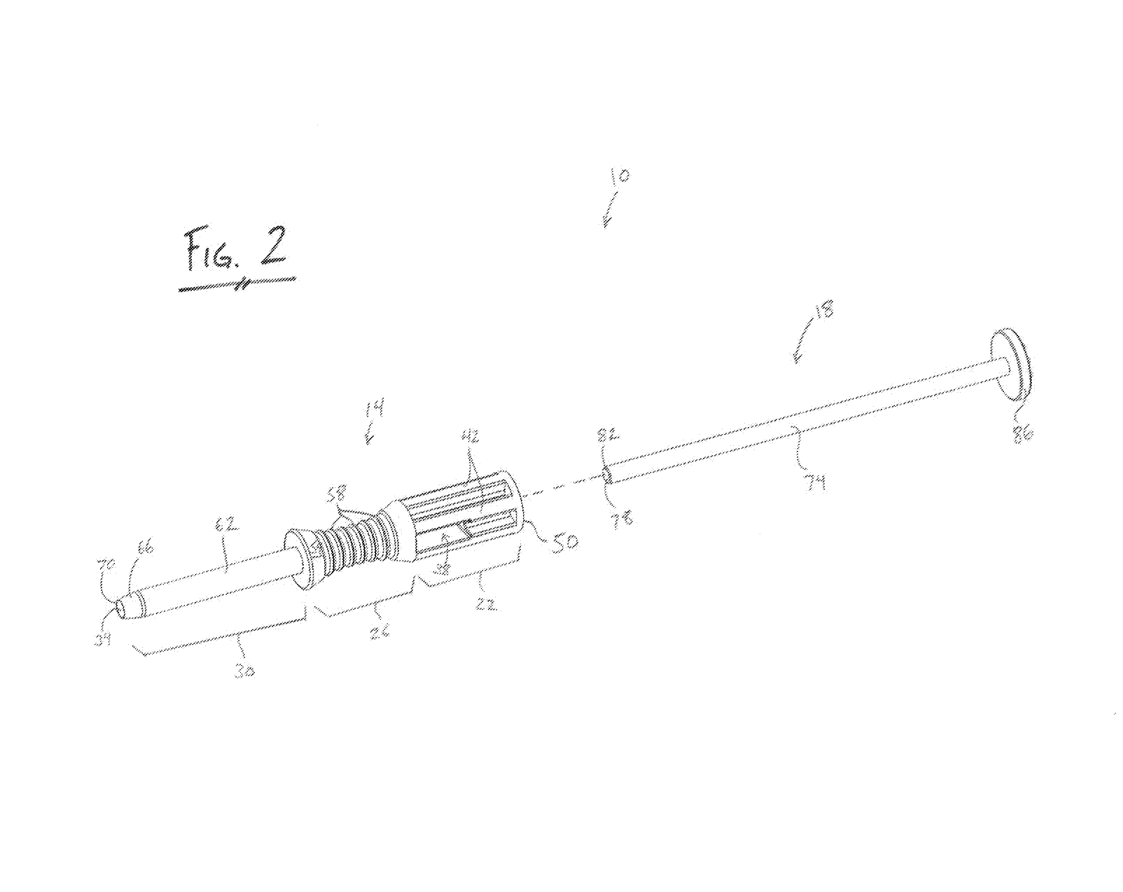

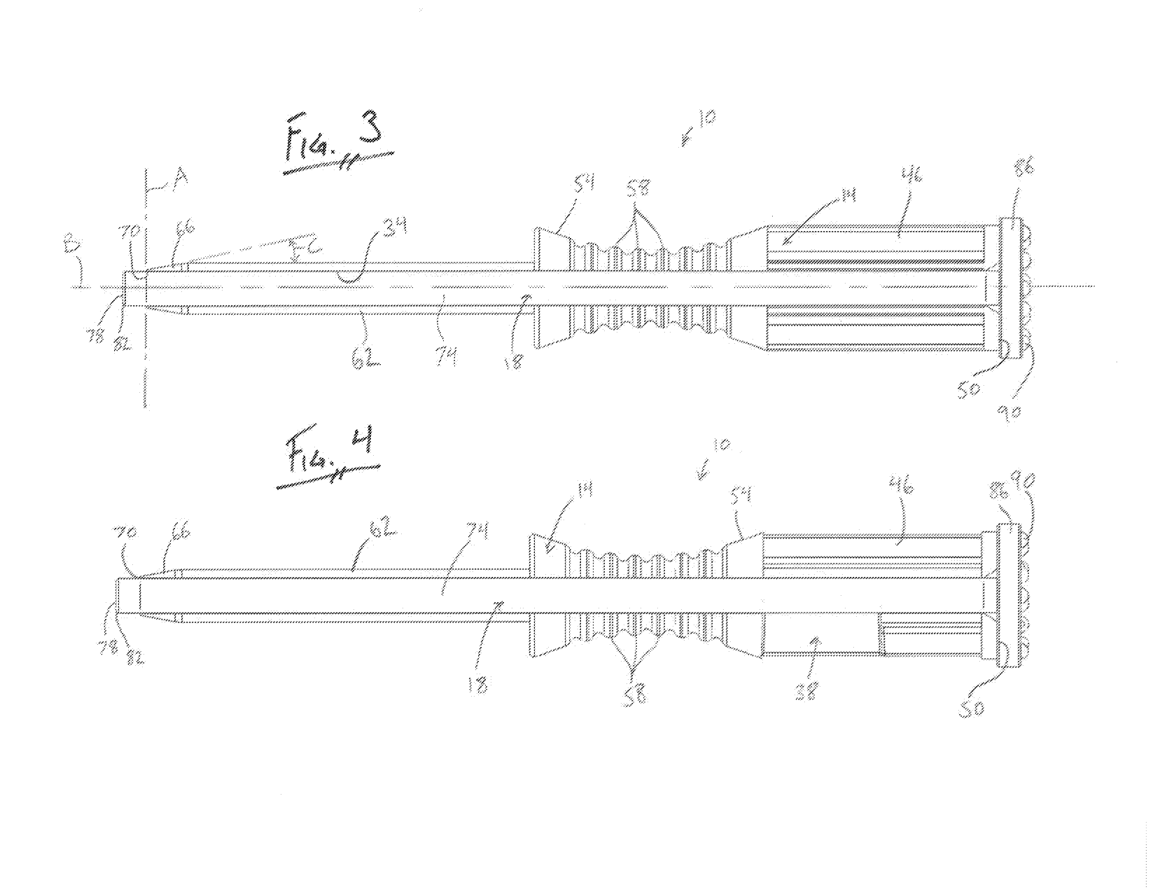

The obturator 18 is shown clearly in FIG. 2 and includes a shaft 74 defining a length sized to extend fully through the lumen 34 from the obturator flange 50 of the body portion 22 to the circular opening 70 of the incising portion 30, and a diameter sized to match the diameter of the lumen 34. The obturator 18 also includes a tip or needle in the form of a blunt tip 78 that includes a fillet 82. A handle 86 is formed at one end of the obturator 18 and sized to abut the obturator shoulder 50 of the body portion 22. The handle 86 includes texture in the form of bumps 90 (see FIGS. 3 and 4) to improve the users grip on the handle 86 during use.

In operation, a patient's skin is prepped with local anesthetic, and a small tunnel or stab wound is made about three millimeters (3mm) in length into the subcutaneous tissue. The trocar 14 is then gripped by the user's forefinger and thumb about the gripping surface 54 of the handle portion 26 and advanced into the wound under pressure from only the fingers using a rotating motion. No significant pressure is applied from the user's forearm during placement. The trocar 14 is advanced only by force from the thumb and forefinger. The result is a more blunt dissection, rather than the sharp cuts of typical devices that can lead to more damage to the subcutaneous tissue as compared to the disclosed trocar 14. The rotational motion enabled by the design of the beveled tip 66 results in less trauma, bleeding, risk of infection, and pain.

With the trocar 14 in place, the loading chamber 38 is loaded with pellets 42 and the obturator 18 is inserted into the lumen 34. The obturator 18 is then advanced by pushing on the handle 86 to dispense the pellets 42.

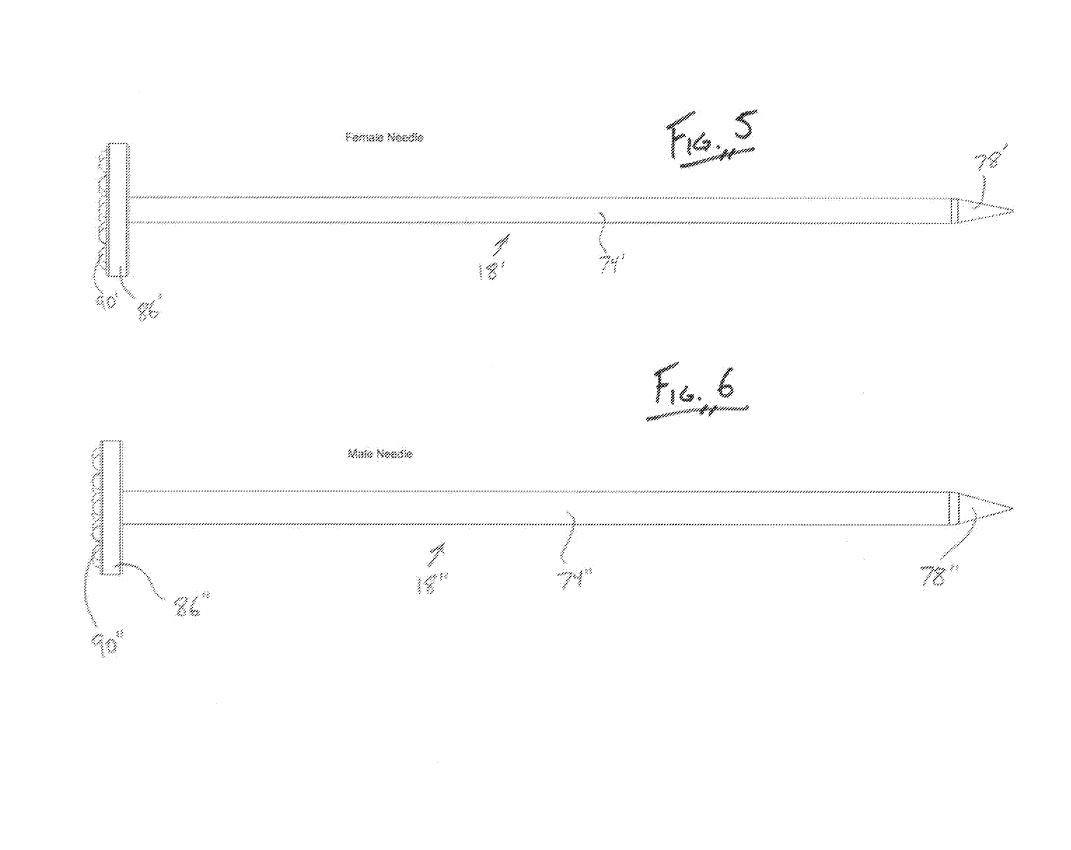

Turning to FIG. 5, a female obturator 18′ is shown and includes a handle 86′ with gripping texture 90′, a shaft 74′, and a tip in the form of a penetrating tip 78′. The female obturator 18′ has a diameter that is smaller than a similar male obturator 18″ shown in FIG. 6, and a female trocar (not shown) and a male trocar (not shown) will include lumens 34 sized to match the diameter of the obturators 18′, and 18″, respectively.

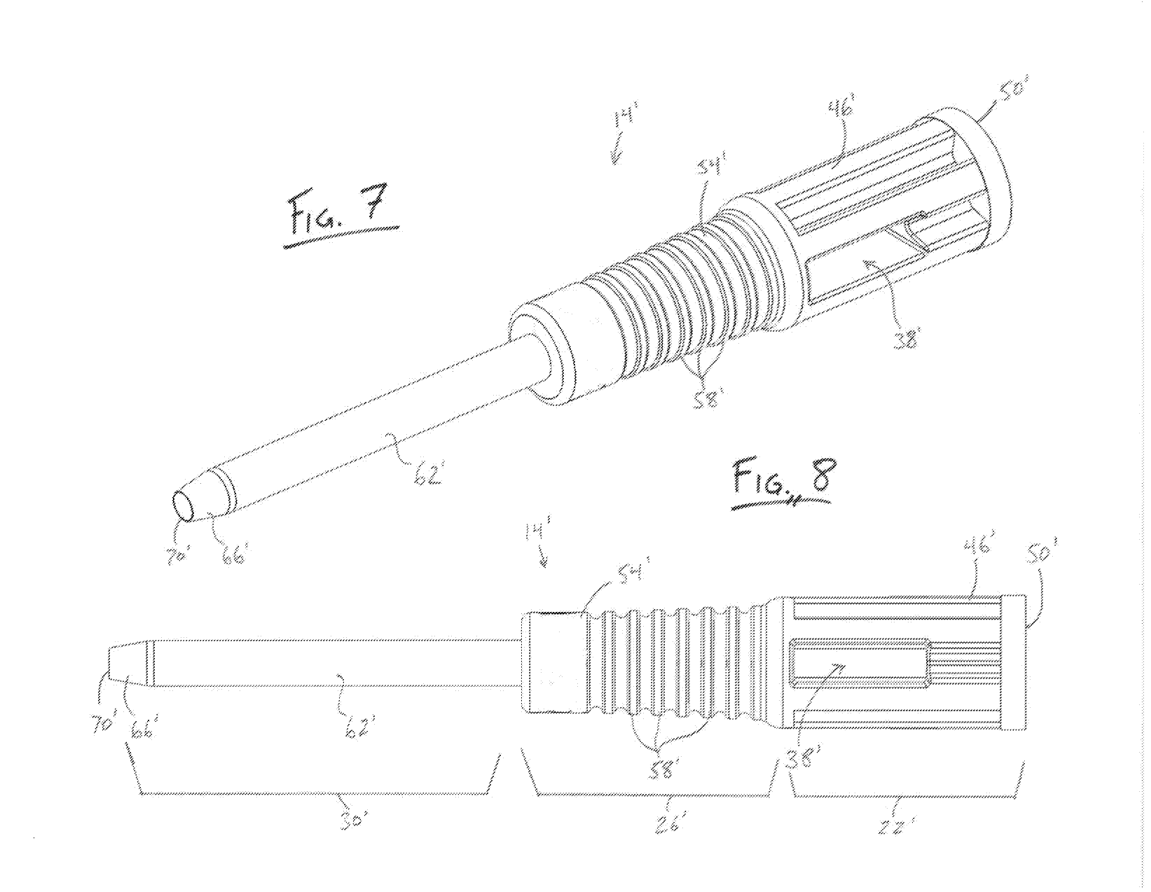

FIGS. 7 and 8 show an alternative trocar 14′ that includes a body portion 22′, a handle portion 26′, and an incising portion 30′. A lumen 34′ is defined through the trocar 14′. The body portion 22′ includes an aperture in the form of a loading chamber 38′ sized to receive a pellet, structural ribs 46′, and an obturator flange 50′. The loading chamber 38′ provides communication to the lumen 34′, such that pellets may be introduced into the lumen 34′ as will be discussed in greater detail below.

The handle portion 26′ includes a gripping surface 54′ with texture in the form of ribs 58′ and defines an ergonomic shape in the form of a profile that tapers, reducing in diameter from the body portion 22′ toward the incising portion 30′ that allows a user to grip the handle portion 26′ during use with less muscle fatigue.

The incising portion 30′ includes a cannula 62′ and a beveled tip 66′ that defines a circular opening 70′ with a cutting surface defined in a plane A′ perpendicular to an axis B′ of the lumen 34′.

The devices described above are preferably made out of a plastic material and preferably produced in a single mold and are produced to be disposable. Alternate embodiments may include materials in the gripping surfaces 54 and 54′ that are designed to promote easier gripping by the user. Exemplary materials include compliant materials such as rubber. For example, the ribs 58 and 58′ may include a rubber material to enhance gripping. The devices discussed above help teach the user doing the insertion to follow the path of the trocar. Typical trocar systems require a straight forward push that requires more force and do not allow for rotation. If the user of the above described devices meets an obstruction, it is possible to manipulate the devices to get around the obstruction. This is possible because of the greater degree of control offered by the handle portions and the arrangement of the beveled tips. Conventional trocars are not easily manipulated and the standard practice is to push through obstructions thereby causing additional tissue damaging and/or bruising and other trauma.

All of the apparatus, methods and algorithms disclosed and claimed herein can be made and executed without undue experimentation in light of the present disclosure. While the invention has been described in terms of preferred embodiments, it will be apparent to those of skill in the art that variations may be applied to the apparatus, methods and sequence of steps of the methods without departing from the concept, spirit and scope of the invention. More specifically, it will be apparent that certain components may be added to, combined with, or substituted for the components described herein while the same or similar results would be achieved. All such similar substitutes and modifications apparent to those skilled in the art are deemed to be within the spirit, scope and concept of the invention as defined.

What is claimed is:

1. A trocar system comprising:
a trocar including a body portion defining a loading chamber, an ergonomically shaped handle portion, and an incising portion that defines a beveled tip, with a lumen formed through the body portion, the handle portion, and the incising portion; and
an obturator including a tip, a shaft, and an obturator handle, the shaft sized to be received within the lumen.

2. The trocar system of claim 1, wherein the handle portion defines a hyperbolic shape.

3. The trocar system of claim 1, wherein the handle portion defines a tapered profile reducing in diameter from the body portion toward the incising portion.

4. The trocar system of claim 1, wherein the handle portion includes a gripping texture.

5. The trocar system of claim 1, wherein the handle portion includes ribs.

6. The trocar system of claim 1, wherein the lumen defines an axis, and
wherein the beveled tip defines a circular opening with a penetrating surface defined in a plane perpendicular to the axis.

7. A method of introducing a hormone pellet into a patient at an implant location using a trocar system that includes
a trocar with a body portion defining a loading chamber, an ergonomically shaped handle portion, an incising portion that defines a beveled tip, and a lumen formed through the body portion, the handle portion, and the incising portion, and
an obturator including a tip, a shaft, and an obturator handle, the shaft sized to be received within the lumen, the method including:
anesthetizing the implant location;
incising the implant location with a wound;
inserting the trocar into the wound using only force from the user's forefinger and thumb and rotating motion;
loading a pellet into the loading chamber;
loading the obturator into the lumen behind the pellet; and
dispensing the pellet into the wound using the obturator.
